SEQUENCE LISTING (17 sequences)

SEQ ID NO: 1 - DNA, 624 bp, Bacillus thuringiensis
accgcttgtgcgggcccccgtcaattcctttgagtttcagccttgcggccgtactccccaggcggagtgcttaatgcgttaacttcagcactaaagggcggaaaccctctaacacttagcactcatcgtttacggcgtggactaccagggtatctaatcctgtttgctccccacgctttcgcgcctcagtgtcagttacagaccagaaagtcgccttcgccactggtgttcctccatatctctacgcatttcaccgctacacatggaattccactttcctcttctgcactcaagtctcccagtttccaatgaccctccacggttgagccgtgggctttcacatcagacttaagaaaccacctgcgcgcgctttacgcccaataattccggataacgcttgccacctacgtattaccgcggctgctggcacgtagttagccgtggctttctggttaggtaccgtcaaggtgccagcttattcaactagcacttgttcttccctaacaacagagttttacgacccgaaagccttcatcactcacgcggcgttgctccgtcagactttcgtccattgcggaagattccctactgctgcctcccgtaggagtctgggccgtgtctcag

SEQ ID NO: 2 - DNA, 731 bp, Bacillus amyloliquefaciens
atgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccc

SEQ ID NO: 3 - DNA, 620 bp, Bacillus thuringiensis
ccacgccgtaaacgatgagtggtaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgt

SEQ ID NO: 4 - DNA, 734 bp, Bacillus subtilis
caagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaaca

SEQ ID NO: 5 - DNA, 689 bp, Bacillus subtilis
caagtcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctc

SEQ ID NO: 6 - DNA, 602 bp, Bacillus subtilis
acagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaag

SEQ ID NO: 7 - DNA, 710 bp, Bacillus subtilis
gggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagt

SEQ ID NO: 8 - DNA, 683 bp, Bacillus amyloliquefaciens
cgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactct

SEQ ID NO: 9 - DNA, 669 bp, Bacillus subtilis
tcgagcggacagatgggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagt

SEQ ID NO: 10 - DNA, 578 bp, Bacillus amyloliquefaciens
aggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcag

SEQ ID NO: 11 - DNA, 682 bp, Bacillus subtilis
gggagcttgctccctgatgttagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaac

SEQ ID NO: 12 - DNA, 636 bp, Bacillus subtilis
gggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaac

SEQ ID NO: 13 - DNA, 695 bp, Bacillus amyloliquefaciens
gggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggag

SEQ ID NO: 14 - DNA, 621 bp, Bacillus amyloliquefaciens
ccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgta

SEQ ID NO: 15 - DNA, 569 bp, Bacillus amyloliquefaciens
cctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactgggga

SEQ ID NO: 16 - DNA, 640 bp, Bacillus amyloliquefaciens
gatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagt

SEQ ID NO: 17 - DNA, 663 bp, Bacillus subtilis
tagcggcggacgggtgagtaacacgtgggtaacctgcctgtaagactgggataactccgggaaaccggggctaataccggatggttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaact

(19) United States
(12) Patent Application Publication
(10) Pub. No.: US 2017/0042949 A1
(43) Pub. Date: Feb. 16, 2017

(54) SYSTEM AND METHOD FOR PRODUCTION OF SHELF STABLE PROBIOTICS FOR ANIMAL NUTRITION ENHANCEMENT

(71) Applicant: THE BOARD OF REGENTS FOR OKLAHOMA STATE UNIVERSITY, Stillwater, OK (US)

(72) Inventors: ALEJANDRO PENALOZA-VAZQUEZ, Stillwater, OK (US); PATRICIA RAYAS-DUARTE, STILLWATER, OK (US)

(21) Appl. No.: 15/303,541

(22) PCT Filed: Apr. 15, 2015
(86) PCT No.: PCT/US2015/025983

§ 371 Date: Oct. 12, 2016

Related U.S. Application Data
(60) Provisional application No. 61/979,766, filed on Apr. 15, 2014.

(51) Int. Cl. A61K 35/742; A23K 10/18; A23K 50/75; A61K 9/00

(52) U.S. Cl. CPC A61K 35/742; A61K 9/0056; A23K 10/18; A23K 50/75

(57) ABSTRACT

Provided are shelf-stable probiotic compositions comprising endospores of Bacillus with desirable characteristics such as acid resistance, high temperature tolerance, and high levels of phytase, α-amylase, cellulolytic and/or protease enzyme activities. Methods for their use, e.g. in food for animal and human consumption, are also provided, as are food products which comprise the probiotics and methods of making the same.

### CROSS REFERENCE TO RELATED APPLICATIONS

This application claims the benefit of U.S. Provisional Patent Application Ser. No. 61/979,766 filed on Apr. 15, 2014, and incorporates said provisional application by reference into this document as if fully set out at this point.

### SEQUENCE LISTING

This application includes as the Sequence Listing the complete contents of the accompanying text file “Sequence.txt”, created Apr. 15, 2015, containing 17,031 bytes, hereby incorporated by reference.

### TECHNICAL FIELD

This disclosure relates to the general subject of probiotics for use in animal feed. In particular, the invention relates to shelf stable probiotic compositions comprising endospores of Bacilli with desirable characteristics such as acid resistance, ox bile resistance, stability at high temperature, particular enzyme activities, etc. and methods for their use, e.g. in animal feed.

### BACKGROUND

Phytic acid (inositol hexakisphosphate (IP6), inositol polyphosphate, or phytate when in salt form), is a saturated cyclic acid, and is the principal storage form of phosphorus in many plant tissues, especially bran and seeds. Ruminant animals are readily able to digest phytate because of the phytase produced by rumen microorganisms. However, phosphorus and inositol in phytate form are not, in general, bioavailable to nonruminant animals because they lack the digestive enzyme phytase required to remove phosphate from the inositol in the phytate molecule. In most commercial agriculture, nonruminant livestock, such as swine, fowl, and fish, are fed mainly grains, such as maize, legumes, and soybeans. Because phytate from these grains and beans is unavailable for absorption, the unabsorbed phytate passes through the gastrointestinal tract, elevating the amount of phosphorus in the manure and potentially leading to environmental problems, such as eutrophication. The lack of absorption and bioavailability negatively impacts host utilization of these essential micronutrients and results in a decrease in growth and weight accumulation of the animals. Such micronutrient deficiencies have been addressed by feeding fortified and biofortified feed stocks, e.g. by providing probiotic supplements, additives and antibiotics. However, the use of antibiotics in particular is increasingly problematic due to growing health concerns due to the evolution of antibiotic-resistant bacteria in animals that consume antibiotics. Alternatives to this practice are needed.

Phytases are presently used as additives in the diet of swine, fisheries, and poultry industrial operations and are proven to improve the availability of phosphorus and vital minerals such as calcium, iron, zinc, etc., resulting in an increased digestibility and gained weight in younger animals (Almeida et al., 2013). Phytases release the phosphorous bound in phytate molecules, avoiding the need for supplementation with inorganic phosphorous, thus reducing the cost of production. Significantly, the excretion of phosphorous in manure in large operations of animal production is also advantageously decreased, thereby decreasing the environmental risk of surface water pollution (Knowlton et al., 2004).

The majority of the commercial feed phytase additives are sourced from Peniophora iycii, Escherichia coli, and Aspergillus niger. However, the present commercial sources of phytases have low thermostability and low specificity (Fu et al., 2008), contributing to shortened shelf-life.

In contrast, the thermostability of Bacillus phytases is well documented and ranges from about 80° to 95° C. Thus, Bacillus phytases tend to survive the pelleting process used in feed preparation, which may include the use of high temperatures and steam (Fu et al., 2008), making Bacillus phytases suitable for commercialization as feed additives. As a result, Bacillus spp. have been used commercially in animal husbandry applications for several years and are among the most widely researched direct-fed microbials to be used as an alternative to antibiotic growth promoters. For example, when fed to broilers, strains of Bacillus spp. have been observed to change the gastro-intestinal microbial profile and contribute to the reduction of pathogens, resulting in health benefits to the host. Although the exact mechanism of improvement of the overall health status, and in particular the intestinal tract, is not well understood, there is a growing body of evidence that supports this occurrence (Alexopoulos et al 2004, Sen et al 2012). For example, studies of direct-fed microbials of Bacillus in poultry have shown significant advantages in increased body weight gain in both chicks and poults (Shivaramaiah et al 2011). Santoso et al (2001) reported increased nutrient digestion and utilization in broiler chicks due to supplementation with a Bacillus direct-fed microbial; the authors speculated that this was due to the secretion of proteases, amylase and lipase by the Bacillus. The literature also reports that competitive exclusion and the probiotic properties of Bacillus in the gastrointestinal tract of poultry and pigs support their overall health (Casula and Cutting 2002, Guo et al 2006). Sen et al (2012) reported that Bacillus subtilis LS 1-2 supported growth in broiler diets and improved intestinal microbial balance and gut health. Probiotics have also been shown to have favorable health effects in humans, and spores of Bacillus species (spp.) are commercially available and used as probiotics and competitive exclusion agents for both animals and humans in several countries including the U.S. (Casula and Cutting 2002, Hong et al 2005).

Many fermented food products that are described as favorable to health also contain Bacilli which produce a variety of desirable metabolites and a number of biologically active compounds. Examples include isolates obtained from fermented soybean foods in China such as natto, yandou, and fermented okara among others (Fan et al., 2013, Qin et al., 2013, Zhu et al., 2008).

One important observation is that Bacillus subtilis germinates and sporulates in the jejunum, and more spores are generally recovered from the feces of hosts than are actually ingested (Tam et al., 2006). Several researchers have suggested that Bacillus are ingested and thrive in the human and animal intestine but do not colonize the intestinal tract, implying a need to continually replenish the supply. The ability to sporulate provides an advantage to the use of Bacillus as probiotics since they therefore have a long shelf life compared to the vegetative organisms Lactobacillus, Bifidobacterium and nonpathogenic yeasts, which are more commonly used as probiotics for livestock and poultry at the present time (Quigley, 2010, Gaggia et al., 2010).

Challenges faced when developing probiotic supplements and products containing those supplements include identifying microbes with high levels of one or more desired enzyme activities (e.g. phytase activity), and which are robust enough to meet the demands associated with probiotic manufacturing and storage needs so that they reach their intended recipients in viable condition. While some feed supplements are currently available which address these issues, there is an ongoing need to provide more effective supplements, especially from natural sources, which can eliminate the use of antibiotics, and which perform better than currently available probiotics. Accordingly, it should now be recognized, as was recognized by the present inventors, that there exists, and has existed for some time, a very real need for an invention that would address and solve the above-described problems.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

High fiber diets which are commonly used in animal feeds are rich in phytate or phytic acid, both of which impair the absorption of nutrients such as minerals and proteins, causing macro/micronutrient deficiencies and decreased growth in hosts that consume them. While current probiotic supplements attempt to address these problems, those that are currently available suffer from a lack of overall desirable enzyme activities and/or from relatively short shelf-lives. This invention solves these problems by providing shelf stable probiotic cocktails containing spores from a combinations of selected Bacillus bacterial species or strains having high levels of selected, desirable enzyme activities, including phytase, protease, cellulase, and amylase. The bacteria and endospores are advantageously acid resistant and stable at high temperatures, and thus the compositions exhibit exceptional stability during manufacturing, storage and use. When ingested by a host, the spores germinate in the intestinal tract and the vegetative Bacilli produce beneficial enzymes which aid in digestion of high fiber diets. Bioavailability of nutrients which would otherwise pass through the hosts digestive system unutilized is increased, and the nutritional status of the host is improved.

According to an embodiment, there is provided herein a probiotic composition comprising endospores of one or more Bacillus species deposited under NRRL accession number B-67039, B-67040, B-67041, B-67042, B-67043, B-67044, B-67045, B-67046, B-67047, B-67048, B-67049, B-67050, B-67051, B-67052, B-67053, B-67054, or B-67055, and a physiologically acceptable carrier.

There is also provided herein an embodiment of a method of increasing nutrient availability from a high fiber diet and digestion efficiency in a host in need thereof, comprising administering to said host a probiotic composition comprising endospores of one or more Bacillus species deposited under NRRL accession number B-67039, B-67040, B-67041, B-67042, B-67043, B-67044, B-67045, B-67046, B-67047, B-67048, B-67049, B-67050, B-67051, B-67052, B-67053, B-67054, or B-67055, and a physiologically acceptable carrier.

There is further provided herein an embodiment of a method of increasing weight gain in an animal in need thereof, comprising administering to said animal a probiotic composition comprising endospores of one or more Bacillus species deposited under NRRL accession number B-67039, B-67040, B-67041, B-67042, B-67043, B-67044, B-67045, B-67046, B-67047, B-67048, B-67049, B-67050, B-67051, B-67052, B-67053, B-67054, or B-67055, and a physiologically acceptable carrier.

There is still further provided herein an embodiment of a probiotic food product comprising endospores of one or more Bacillus species deposited under NRRL accession number B-67039, B-67040, B-67041, B-67042, B-67043, B-67044, B-67045, B-67046, B-67047, B-67048, B-67049, B-67050, B-67051, B-67052, B-67053, B-67054, or B-67055.

There is still further provided herein an embodiment of a method of making a probiotic food product comprising combining endospores of one or more Bacillus species deposited under NRRL accession number B-67039, B-67040, B-67041, B-67042, B-67043, B-67044, B-67045, B-67046, B-67047, B-67048, B-67049, B-67050, B-67051, B-67052, B-67053, B-67054, or B-67055, with one or more additional nutritional components or carriers.

The foregoing has outlined in broad terms some of the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1 contains a schematic illustration of the initial steps in the method of isolating sporulating bacteria from sourdough.

FIG. 2 contains a schematic illustration of subsequent steps in the method of isolating sporulating bacteria from sourdough.

FIG. 3 contains a table that describes exemplary colony morphology criteria.

FIGS. 4A and B contains photographs showing examples of characteristic colony morphology of A, Bacillus thuringiensis and B, Bacillus subtillus.

FIG. 5A-D contains photographs showing examples of bacterial strains that exhibit A, phytase activity, B, α-amylase activity, C, cellulolytic activity, and D, protease activity.

FIG. 6 contains a summary of data obtained from the testing of 30 colonies grown on LB plus ox bile from 0.3 to 4% with respect to phytase, α-amylase, cellulolase (cellulolytic) and protease activity.

FIG. 7. Spore count in colony forming units (CFU)/mL as a function of incubation time of six Bacillus spp. strains from the OSU collection.

FIG. 8. Example of survival of spores from strains 3, 6, 19 and 24 in water after 92 h.

FIG. 9. Example of growth curve in 10 L fermenter of OSU 3 strain. OD=optical density.

FIG. 10. Feed Conversion Ratio (FCR) of Bacillus spores from three strains used as probiotics in male Cobb500 broilers.

FIG. 11A-Q. DNA sequences encoding 16sRNA for Bacilli deposited under NRLL accessions numbers: A, B-67039; B, B-67040; C, B-67041; D, B-67042; E, B-67043; F, B-67044; G, B-67045; H, B-67046; I, B-67047; J, B-67048; K, B-67049; L, B-67050; M, B-67051; N, B-67052; O, B-67053; P, B-67054; and Q, B-67055.

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

In some aspects, the invention provides probiotic compositions comprising microorganisms with high activity for biodegradation of phytic acid, as well as methods of producing and using the compositions. Accordingly, the invention addresses macro/micronutrient deficiencies, e.g. in monogastric hosts with high fiber diets, and provides direct improvements to human and animal nutrition. When ingested, the probiotic compositions enhance the nutritional value of high fiber diets by increasing the availability of minerals and proteins. In addition, the compositions, especially when ingested on a regular basis, may serve to eliminate or decrease the growth of undesirable pathogenic microbes by competing for gut resources.

The probiotic compositions comprise Bacillus species or strains, generally in the form of endospores, that exhibit high levels of desirable enzyme activities, acid resistance, temperature stability (and thus long shelf lives), and yet are relatively inexpensive to produce and maintain. The Bacilli are advantageously derived from natural sources. In fact, the Bacilli were isolated from whole wheat sourdough. Investigations characterizing these probiotic species, including enzyme efficiencies, spore generation, stability and impact on carcass composition of broilers which consume them, are described for the first time herein.

The following definitions and terms are used throughout:

Probiotics are generally understood to be live microbial food supplements that can benefit the host by improving its intestinal balance and/or nutrient availability.

Bacillus is a genus of Gram-positive, rod-shaped, bacteria and a member of the phylum Firmicutes. Bacillus species can be obligate aerobes (oxygen reliant), or facultative anaerobes (having the ability to be aerobic or anaerobic). They test positive for the enzyme catalase in the presence of oxygen. Under stressful environmental conditions, the bacteria produce oval endospores. “Endospores” are not true spores, but represent in a dormant state to which the bacteria can reduce themselves, and in which they remain viable for long periods of time.

An endospore is a dormant, tough, and non-reproductive (non-vegetative) structure produced by certain bacteria from the Firmicute phylum, such as Bacillus. The name “endospore” is suggestive of a spore or seed-like form (endo means within), but it is not a true spore i.e., endospores are not offspring. Rather, an endospore is a stripped-down, dormant form to which the bacterium can reduce itself. However, the terms “spore” and “endospore” may be used interchangeably herein. Endospore formation is usually triggered by a lack of nutrients. In endospore formation, the bacterium divides within its cell wall and one side then engulfs the other. The endospore itself comprises the bacterium's DNA, ribosomes and large amounts (up to 10% of the endospore's dry weight) of dipicolinic acid which appears to help in the ability of endospores to maintain dormancy. Endospores can survive without nutrients and are resistant to ultraviolet radiation, desiccation, high temperature, extreme freezing and chemical disinfectants. Endospores enable bacteria to lie dormant for extended periods, even centuries. When the environment becomes more favorable, the endospore can reactivate itself to the vegetative state.

Provided herein are probiotic compositions comprising endospores from at least one Bacillus species or strain as described herein. In some aspects, the probiotics comprise endospores of Bacillus species and/or strains deposited as NRRL accession numbers NRRL B-67039, B-67040, B-67041, B-67042, B-67043, B-67044, B-67045, B-67046, B-67047, B-67048, B-67049, B-67050, B-67051, B-67052, B-67053, B-67054, and B-67055. Table 1 shows the informal, internal designations of each Bacillus, the genus and species of each, the NRLL deposit number of each, and the SEQ ID NO: that corresponds to DNA that encodes the 16S ribosomal RNA (16S rRNA) for each Bacillus. The invention also encompasses a Bacillus (or offspring thereof) having all the characteristics of a Bacillus deposited as any one of the accession numbers listed in Table 1, and combinations thereof comprising 2, 3, 4, 5, 6, 7, 8, 9, 10, 11, 12, 13, 14, 15, 16 or all 17 of the Bacilli. The combinations may be in any of the various forms discussed herein, e.g. probiotic supplements, food products, etc. The food product is not a sourdough. The SEQ ID NOS: which correspond to DNA encoding the 16S rRNA for each of the Bacilli are also provided. Those DNA sequences and the 16S rRNA encoded by them are also encompassed by the invention, as are Bacilli having (comprising) the DNA sequences and/or 16S rRNA encoded by the DNA sequences. Probiotics as described herein may comprise at least one and generally comprise two or more different types of Bacilli with DNA having a sequence as set forth in one of SEQ ID NOS: 1-17, and/or having 16S rRNA encoded by such a DNA sequence.

TABLE 1
For 17 exemplary Bacillus strains, informal internal designations, genus
and species, NRLL accession numbers and SEQ ID NOS: corresponding
to DNA that encodes 16S rRNA for each

| | | | SEQ ID NO: |
|---|---|---|---|
| | | | of DNA that |
| | Microorganism identified | | encodes |
| | by complete 16S rRNA | Registration | corresponding |
| Strain ID | gene sequence | Number | 16sRNA |
| OSU 2 | Bacillus thuringiensis | NRRL B-67039 | 1 |
| OSU 3 | Bacillus amyloliquefaciens | NRRL B-67040 | 2 |
| OSU 5 | Bacillus thuringiensis | NRRL B-67041 | 3 |
| OSU 6 | Bacillus subtilis | NRRL B-67042 | 4 |
| OSU 7 | Bacillus subtilis | NRRL B-67043 | 5 |
| OSU 9 | Bacillus subtilis | NRRL B-67044 | 6 |
| OSU 10 | Bacillus subtilis | NRRL B-67045 | 7 |
| OSU 11 | Bacillus amyloliquefaciens | NRRL B-67046 | 8 |
| OSU 13 | Bacillus subtilis | NRRL B-67047 | 9 |
| OSU 19 | Bacillus amyloliquefaciens | NRRL B-67048 | 10 |
| OSU 20 | Bacillus subtilis | NRRL B-67049 | 11 |
| OSU 21 | Bacillus subtilis | NRRL B-67050 | 12 |
| OSU 23 | Bacillus amyloliquefaciens | NRRL B-67051 | 13 |
| OSU 24 | Bacillus amyloliquefaciens | NRRL B-67052 | 14 |
| OSU 25 | Bacillus amyloliquefaciens | NRRL B-67053 | 15 |
| OSU 28 | Bacillus amyloliquefaciens | NRRL B-67054 | 16 |
| OSU 29 | Bacillus subtilis | NRRL B-67055 | 17 |

In some aspects, each of the Bacillus species or strains has a high level of at least one enzyme activity of interest, and a combination of the strains/species exhibits at least one, and usually 2, 3, 4, 5, 6, etc. or more enzyme activities of interest. The Bacillus species or strains which have been selected have also been chosen to minimize unfavorable interactions with each other, i.e. they are compatible with each other in that generally, each species or strain does not inhibit the growth of or expression of enzyme activity of any other species or strain in the mixture, when grown in proximity to each other, e.g. when grown in mixed cultures. Further, they are generally heat tolerant and acid resistant. These characteristics are discussed in greater detail below.

Generally, the enzymatic activity of interest is associated one or more enzymes which participate in (e.g. catalyze) the breakdown (digestion) of at least one component of a high fiber diet. In some aspects, the enzyme activity is one or more of phytase activity (e.g. breakdown of phytate and phytic acid), protease activity (hydrolysis of proteins, polypeptides and peptides); amylase activity (such as e.g. α-amylase activity, which breaks down starches to simple sugars such as fructose, maltose, glucose, etc.); and cellulolytic activity (e.g. digestion of cellulose).

In addition, the probiotic mixtures are advantageously stable (particularly when they comprise endospores), and they have long shelf lives. For example, they are resistant to high temperatures and remain viable (e.g. remain in or are able to return to a vegetative state) when processed at 70, 75, 80, 85, 90, or 95° C. or stored at temperatures ranging from about 26° C. to about 45° C. for up to about one year. These temperatures accord with those used during animal feed production, and thus when probiotics of the invention are mixed with animal feed components, their associated enzyme activities are not decreased or attenuated during processing.

The components of the probiotic mixture are also acid bile acid resistant. For example, the Bacilli display excellent growth even when grown in or on medium comprising 4% ox bile. Bile acid resistance is advantageous because it enables the Bacilli to survive and grow in the intestinal tract of animals.

The shelf life of a probiotic composition, whether as a composition per se, or when combined with other ingredients such as animal feeds, is advantageously long. For example, the Bacilli in the composition remain viable for periods of time up to at least about one year, when stored, for example, at about 45° C.

The Bacilli and/or endospores that are included in the probiotic compositions described were isolated from whole wheat sourdough. Each Bacillus was isolated and obtained in substantially pure form, e.g. as a single colony isolate from which cultures were grown, tested and maintained. Particular cultures were selected for acid resistance and ox bile resistance to 4%, heat tolerance, and the ability to carry out at least one particular enzyme activity. As such, the individual cultures are not natural products, since the bacteria would not occur in purified form in nature. Further, when used in the practice of the invention, combinations of two or more bacteria are combined into a single mixture so that, in some embodiments, the mixture consists essentially of two or more of the characterized bacteria and/or endospores thereof as the living components. That is, the portion of the mixture that contains the biologically active bacteria or endospores consists essentially of two or more of the characterized bacteria and/or endospores. Other components present in the mixture include, for example, carriers, various non-living media components, salts, nutrients, buffers, etc. that are necessary or beneficial to maintenance of the bacteria and/or endospores.

The present invention provides compositions for delivering a probiotic mixture to a subject. In some aspects, such as in compositions that are “cocktails”, endospores from 2, and possibly 3, 4, 5, or 6 or more (e.g. about 10, 15 or 20) strains are combined in a probiotic composition. The compositions generally also include a suitable carrier that is physiologically compatible. The compositions are prepared as liquids (e.g. solutions or suspensions, which may be in concentrated e.g. 10× or 100× form), or in solid forms such as capsules, tablets, pills, powders, granules and the like. Solid forms suitable for solution in, or suspension in, liquids prior to administration may also be prepared. The preparation may also be emulsified. The liquids may be aqueous or oil-based suspensions or solutions. The probiotic may be in the form of a paste or gel. The active ingredients may be mixed with excipients which are physiologically acceptable and compatible with the endospores such as, for example, water, saline, saccharides, and the like, or combinations thereof. In addition, the composition may contain minor amounts of auxiliary substances such as wetting or emulsifying agents, pH buffering agents, and the like. In addition, the composition may contain preservatives. For forms of the probiotics that are administered orally, various thickeners, flavorings, diluents, emulsifiers, dispersing aids or binders and the like may be added. The composition of the present invention may contain any such additional ingredients so as to provide the composition in a form suitable for ingestion. The final amount of endospores in the formulations may vary. However, in general, the amount in the formulations will be from about 1-99%.

Some examples of materials which can serve as acceptable carriers or formulation components include, but are not limited to, ion exchangers, alumina, aluminum stearate, lecithin, various proteins, buffer substances (e.g. phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; and phosphate buffer solutions, as well as other non-toxic compatible lubricants such as sodium lauryl sulfate and magnesium stearate; coloring agents; releasing agents; coating agents; sweetening and flavoring agents, etc. Preservatives and antioxidants can also be present in the composition, according to the judgment of the formulator. Additional beneficial and physiologically active substances may also be included in formulations, such as beneficial bacteria (other types of Bacilli, various Lactobacilli, etc.), vitamins, minerals, carotenoids, physiologically acceptable salts, amino acids, etc. The compositions may contain other active agents such as long chain fatty acids and zinc. Suitable long chain fatty acids include alpha-linoleic acid, gamma linolenic acid, linoleic acid, eicosapentanoic acid, and docosahexanoic acid. Fish oils are a suitable source of eicosapentanoic acids and docosahexanoic acid. Borage oil, blackcurrent seed oil and evening primrose oil are suitable sources of gamma linolenic acid. Safflower oils, sunflower oils, corn oils and soy bean oils are suitable sources of linoleic acid. Zinc may be provided in various suitable forms, for example as zinc sulfate or zinc oxide.

The probiotic may be administered or delivered in any of a variety of suitable ways. For example, it may be administered directly to a subject, e.g. as a liquid drink or in drinking water or another comestible liquid; or as a solid, e.g. in a pill or capsule, the exterior or which is biodegraded thereby releasing the spores into the intestinal tract of the host; or as granules that are sprinkled in or on food or a liquid; or incorporated into or coated onto the surface of an edible substance such as kibble, chow, food pellets, etc.; or as a spray or drops that can be used to coat an edible substance that serves as a carrier of the probiotic. The probiotic may be formulated in any manner that allows its successful use. The invention also comprises concentrates of the compositions described herein.

The probiotic compositions may be part of the normal dietary intake, and/or a supplement. As such, the invention also encompasses foods and edible (ingestible, comestible, etc.) products or items suitable for administration to an animal in need thereof or which could benefit from administration. The food product is not sourdough. The compositions include, e.g. those whose principle component is the probiotic (e.g. a supplement), and those which comprise part of the normal dietary intake and are formulated with the probiotic, but which comprise other nutritional ingredients, including without limitation kibble, chow, food pellets, mash, biscuits, a processed grain feed, a wet animal food, yogurts, gravies, chews, treats, etc.

Other components may be included that are beneficial for inclusion in the compositions used herein, but are optional for purposes of the invention. For example, food compositions are preferably nutritionally balanced. In one embodiment, the food compositions may comprise, on a dry matter basis, from about 20% to about 50% crude protein, preferably from about 22% to about 40% crude protein, by weight of the food composition. The crude protein material may comprise any material having a protein content of at least about 15% by weight, non-limiting examples of which include vegetable proteins such as soybean, cotton seed, and peanut, animal proteins such as casein, albumin, and meat tissue. Non-limiting examples of meat tissue useful herein include fresh meat, and dried or rendered meals such as fish meal, poultry meal, meat meal, bone meal and the like. Other types of suitable crude protein sources include wheat gluten or corn gluten, and proteins extracted from microbial sources such as yeast.

Furthermore, the food compositions may comprise, on a dry matter basis, from about 5% to about 35% fat, preferably from about 10% to about 30% fat, by weight of the food composition. Further still, food compositions comprising the lactic acid bacteria of the present invention may also comprise from about 4% to about 25% total dietary fiber. The compositions may also comprise a multiple starch sources.

The compositions of the present invention may further comprise a source of carbohydrate. Grains or cereals such as rice, corn, milo, sorghum, barley, alfalfa, wheat, and the like are illustrative sources. In addition, the compositions may also contain other materials such as dried whey and other dairy by products.

The compositions comprising the bacteria of the present invention may also comprise a prebiotic. “Prebiotic” includes substances or compounds that are fermented by the intestinal flora of the pet and hence promote the growth or development of lactic acid bacteria in the gastro-intestinal tract of the pet at the expense of pathogenic bacteria. The result of this fermentation is a release of fatty acids, in particular short-chain fatty acids in the colon. This has the effect of reducing the pH value in the colon. Non-limiting examples of suitable prebiotics include oligosaccharides, such as inulin and its hydrolysis products commonly known as fructooligosaccharides, galacto-oligosaccarides, xylo-oligosaccharides or oligo derivatives of starch. The prebiotics may be provided in any suitable form. For example, the prebiotic may be provided in the form of plant material which contains the fiber. Suitable plant materials include asparagus, artichokes, onions, wheat or chicory, or residues of these plant materials. Alternatively, the prebiotic fiber may be provided as an inulin extract, for example extracts from chicory are suitable. Alternatively, the fiber may be in the form of a fructooligosaccharide. Otherwise, the fructooligosaccharides may be obtained by hydrolyzing inulin, by enzymatic methods, or by using micro-organisms.

The compositions may be formulated for particular stages of life or development, such as newborn, juvenile, adult, older adult, etc., and/or for particular conditions or diseases such as diabetes, obesity, dental conditions, exposure to stress, etc. as described elsewhere herein. For example, for chickens, particular preparations with specified or optimized amounts of protein may be made for e.g. grower, intermediate, and finisher phases of growth.

Administration or delivery of the probiotic is generally oral e.g. by mouth. Delivery may be active, i.e. a quantity or dose the probiotic formulation is purposefully administered to individual substances, e.g. by a human caretaker; or delivery may be passive or volitional in nature in that the subject eats the probiotic formulation of its own accord, ingesting either the probiotic per se, or ingesting the probiotic in combination with another food substance with which it has been combined as described elsewhere.

Various formulations and modes of delivery of probiotics are known in the art and may be employed with the probiotic compositions described herein, including those described in U.S. Pat. Nos. 8,993,017, 8,968,721, 8,900,623, 8,871,266 and 8,846,082, the complete contents of each of which are hereby incorporated by reference.

To be effective, doses of probiotic in the range of from at least about 10⁹ to about 10¹⁵ endospores per kg of feed should be ingested. The frequency of administration or delivery may vary and may be, for example once or twice per day, one per week, etc. In some aspects, delivery is not regimented in that the probiotic is comprised in a subject's feed and is simply ingested when the subject eats.

The compositions may be formulated by any acceptable process. For dried animal foods, a suitable process is, for example, extrusion cooking, although baking and other suitable processes may be used. When extrusion cooked, the dried food is usually provided in the form of a kibble or pellets. If a prebiotic is used, the prebiotic may be admixed with the other ingredients of the dried pet food prior to processing. For wet foods, the processes described in U.S. Pat. Nos. 4,781,939 and 5,132,137, the complete contents of which are incorporated by reference in entirety, may be used as may, for example: cooking in a steam oven; by gelling an emulsion; etc.

Animals that may benefit from ingesting the probiotics described herein include, without limitation: primates such as homids (e.g. humans, chimpanzees, gorillas and orangutans); haplorhine (“dry-nosed”) and strepsirrhine (“wet-nosed”) primates; various avian species such as chickens, ducks, geese, ostriches, turkeys and other fowl; fish, e.g. catfish, tilapia, salmon, etc.; crustaceans e.g. lobsters, shrimp, etc.; Equidae such as horses, donkeys, and zebras, etc.; Leporidae such as rabbits; bears; the rhinoceros; the hippopotamus; elephants; rodents such as mice and rats, etc.; reptiles; amphibians; canids such as dogs, wolves, coyotes, etc.; felines such as cats, lynx, bobcats, lions, tigers, cougars, leopards, etc.; the Suidae family of even-toed ungulates such as pigs, Eurasiwild boars, the peccary, babirusa, and warthog; etc. The animals may be monogastric but ruminants may also benefit from the probiotics of the invention. The animals may be commercially raised (for food, sport, etc.), or may be companion pets, or may be located on reserves or in zoos, in animal shelters, etc. The animals may or may not have primarily a high fiber diet, but generally they do consume at least one high fiber food, e.g. grasses, leaves, seeds, legumes, soy beans, fruits, vegetables, etc. The animals may be at any life stage, e.g. newborn, juvenile, adult, during pregnancy, older adult, etc., and/or may need special diets due to other factors such as disease, recovery from disease, various conditions such as diabetes, propensity to urinary tract infections, dental problems, obesity, lack of weight gain, exposure to stress, etc.

The benefits accrue by ingestion of the probiotic supplements. For example, many aspects of health are improved due to the improvement of digestion and nutrient utilization. For example, the growth efficiency and weight gain of e.g. broilers and other animals or fish that are raised commercially for food, are increased, thereby increasing meat production. Another contribution is an improvement in overall health of probiotic recipients due to competition with other microbes, which can eliminate or decrease the number or amount of less favorable microflora, thus favorably balancing gut microflora. The occurrence of disease is thus lessened.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Still further, additional aspects of the instant invention may be found in one or more appendices attached hereto and/or filed herewith, the disclosures of which are incorporated herein by reference as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

### Examples

### Example 1

### Production and Evaluation of Bacillus Spp. as Probiotics for Animal Nutrition Enhancement

Phytic acid is a primary storage form of phosphate in plants. Eighty percent of the phosphorus in seeds and cereals is present as a salt form of phytic acid known as phytate. Phytate's highly negatively charged groups bind tightly with cation groups on protein, amino acids, starch and lipids in feed/foodstuff, reducing the digestibility of these nutrients for consumers such as in fish, poultry, pigs and humans. In order to improve availability of macro/micronutrients, exogenous phytase enzymes can be added to swine, poultry and fish feeds, leading to improved availability of phosphorus, minerals, amino acids, and energy. Studies with infant cereals treated with phytase have also demonstrated increased iron and zinc availability in vitro.

A more desirable means of providing phytase activity is via the use of probiotics, since they also provide a host of other benefits. Probiotics have been shown to promote a variety of biological effects in a number of physiological conditions and pathologies, including allergies, intestinal and liver diseases, urinary and upper respiratory infections, and metabolic diseases. They have been used as live microorganisms and as dietary supplements to promote and sustain health benefits in animals and humans.

A recent study determined the effects of protease and phytase (commercial sources) and a Bacillus spp. direct-fed microbial on dietary energy and nutrient utilization in broiler chickens (Ganapathi et al 2014). It was concluded that significant interaction (P≦0.01) between the two enzymes protease and phytase and Bacillus spp. on the apparent ileal digestibility coefficient for starch, crude protein, and amino acid indicated that both additives increased the digestibility.

Bacillus spores offer many advantages as probiotics compared to, for example, Lactobacillus live cells. The main advantage of Bacillus spores is their thermostability. This property gives a tremendous advantage and competitive edge for delivering probiotic activity with increased shelf life over a wide range of temperatures and resistance to production processes, and preparation advantages.

There is evidence that the Bacillus spores germinate in the intestinal tract of chickens, rats, pigs and humans, although studies are needed to explore the direct comparison of specific strains. For example, it is not known if there are differences in the germination rate of different strains of Bacillus in the intestinal tract. As expected, several researchers have suggested that a number of probiotic properties are strain specific and thus different strains need to be studied in comparative studies under similar conditions to determine which are the most efficacious.

This study contributes to the knowledge of the efficacy of pre-screened and selected Bacillus strains that support the overall wellbeing of broiler chickens and improved utilization of diet nutrients. In the present work, Bacillus spp. were used as one or a mixture of Bacillus strains and no commercial enzymes were included. The specific Bacillus spp. identified herein improve the digestibility and thus nutrient absorption in animals. Supplementing feed ratios with Bacillus spp. spores to a basal diet in male broilers resulted in an improvement in feed conversion and contributed to the overall gut health of the broilers.

### Materials and Methods

Sporulating bacteria were isolated from a batch of whole wheat sourdough as illustrated in FIG. 1. Briefly, LB broth amended with either wheat bran or rice bran (1% (v/v)) was inoculated with a sample of the whole wheat sourdough. Inoculated flasks were incubated at 37° C. for 3-4 days. This step was repeated 4 times and after the 4th time the inoculated medium was centrifuged at 170 rpm for 10 minutes. Pelleted samples were resuspended in 10 ml of sterile water and then kept at 80° C. for 30 minutes to select for heat resistant spores. These conditions typically eliminate all the vegetative cells and select sporulated bacteria like Bacillus spp.

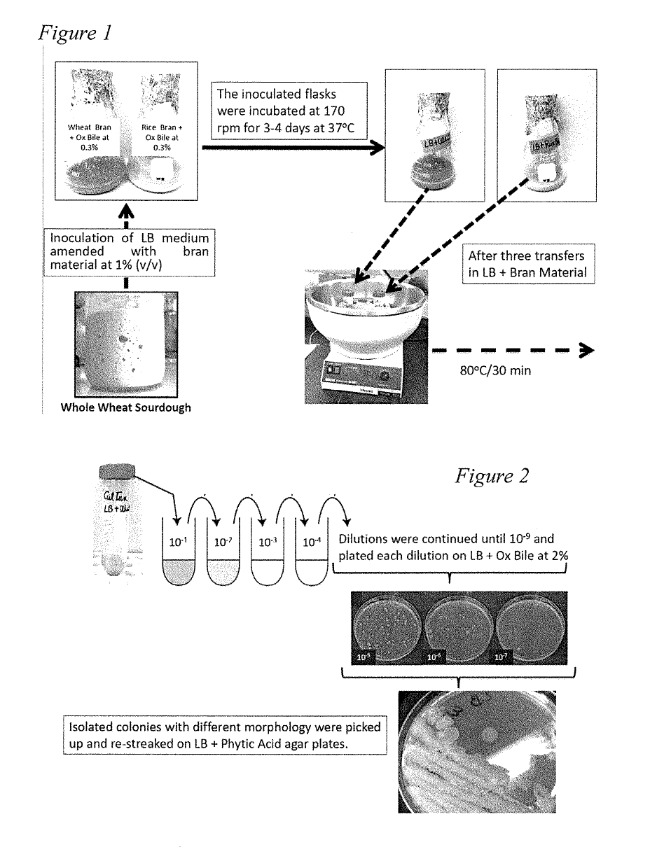

Then, heat shock serial dilutions were performed and plated on LB medium agar amended with phytic acid and ox bile at 4% (LBPHOX) to do standard plate counts of sporulated bacteria and at the same time obtain individual bacterial colonies. Each individual colony was streaked on plates of LBPHOX medium (FIG. 2). Total counts per each of 3 plates for the LB+Rice bran sample were 2.3E+04, 2.00E+04 and 2.00E+03, averaging 1.50E+04 (standard deviation 1.14E+04). Total counts of colony forming units (CFU) for the LB+Wheat bran sample were 2.00E+06, 3.00E+06 and 2.00E+06, averaging 2.33E+06 (standard deviation 5.77E+05).

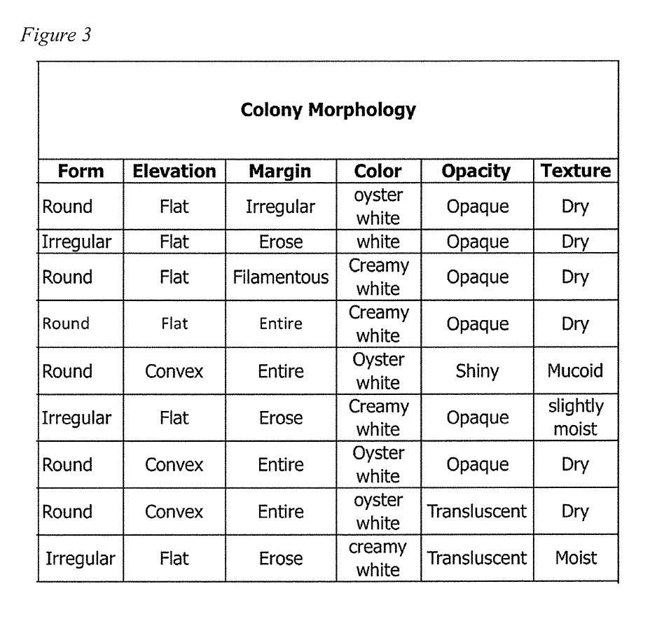

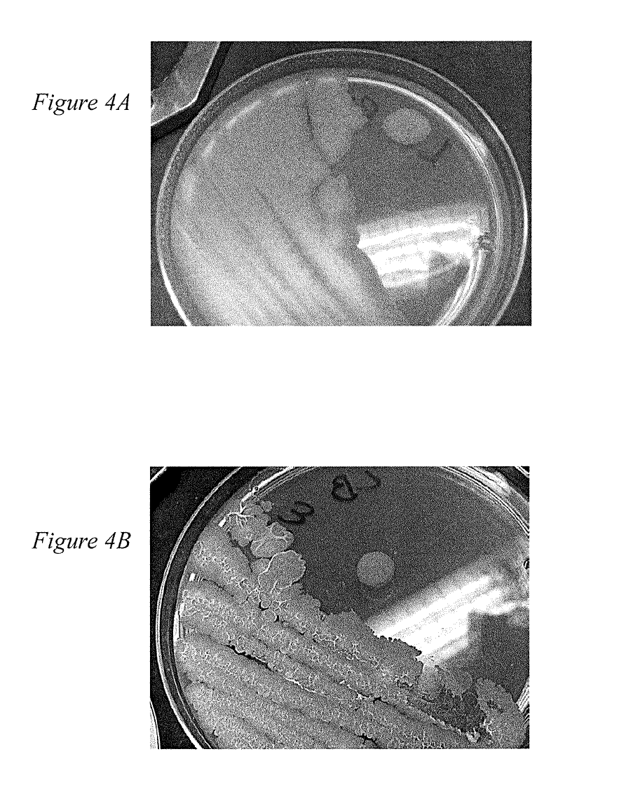

Individual colonies were selected from the plates according to typical Bacillus spp. colony morphology, taking into account the shape (e.g. form, margin and elevation), the color, opacity and texture of the colonies (see FIG. 3). Bacillus spp. colonies in general have the following morphology: shape and size irregular, large; margin undulated (wavy); elevation umbonated or convex; color white, dull; texture dry (or rough) or wrinkled. Exemplary colony morphologies characteristic of Bacillus thuringiensis and Bacillus subtillus are shown in FIGS. 4 A and B. From the selected colonies, the cell morphology and Gram staining were the final criteria for selection: Gram stain positive, cell shape Bacillus arrangement in chains or single, and endospore formers.

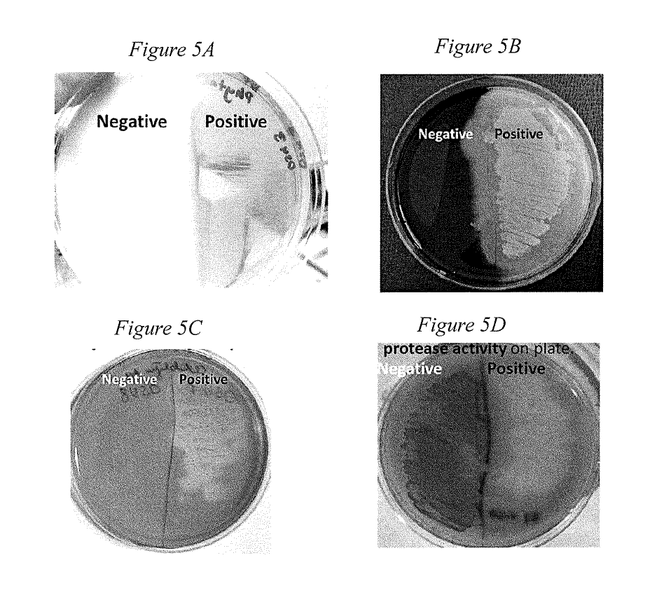

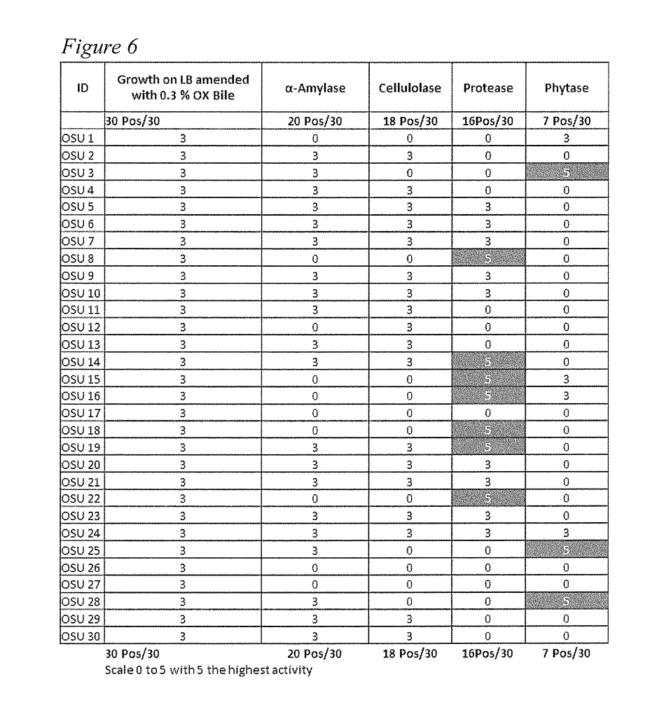

Selected colonies were restreaked on LB+phytic acid agar plates, and incubated at 37° C. for 12 hours. All experiments were performed in triplicate. Plates were examined for zones of phytate hydrolysis around individual colonies, and colonies were further tested as follows: 1) for phytase activity, by cobalt chloride and ammonium molybdate/ammonium vanadate solution (Bae H. D. et al. 1999, Journal of Microbiological Methods, 39:1, 17-22); 2) for α-amylase activity, by the method of Ghani M. et al. 2013, Pakistan Journal of Pharmaceutical Sciences; 26:4, 691-697; 3) for cellulolytic activity using Congo red, also as described by Ghani 2013; and 4) for protease activity, by Coomassie Brilliant Blue R250, destaining with 40% methanol and 10% acetic acid, also as described by Ghani 2013. Exemplary plates showing positive phytase, α-amylase, celluylolytic, and protease activity are shown in FIG. 5A-D. Systematic selections of Bacillus spp. were made according to enzymatic activities of interest as follows: 7 strains were selected as exhibiting phytase activity; 20 strains were selected as exhibiting α-amylase activity; 18 strains were selected as exhibiting cellulolytic; and 16 strains were selected as exhibiting protease activities. A summary of the results obtained with the 30 strains is presented in FIG. 6. As can be seen, several strains exhibited very high protease and phytase activity (e.g. 5 on a scale of 0 to 5, with 5 being the highest activity), several exhibited medium activity for one or more activities (e.g. 3 on the scale) which others exhibited no activity for a given enzyme, and some strains exhibited more than one activity at a medium to high level. From this set of 30 strains, 17 strains were selected as possessing the following criteria: Bacillus Gram positive, endospore formers, acid tolerance, ox bile resistance and production of at least one extracellular enzyme such as phytase, amylase, etc. as described herein. A complete 16S rRNA gene sequence analysis was conducted for the 17 selected strains with the aim of identifying their genus and species, and, utilizing the international classification schemes for microorganisms based on their biological risks, strains of harmless microorganisms (EFB class 1) were selected, EFB class 1 organisms are microorganisms that have never been identified as causative agents of disease in man and that offer no threat to the environment.

### Example 2

### Scale-Up and Testing of Six Selected Strains

Large scale preparation of the six strains was scaled up from 500 mL and 1 liter flask to 10 liter fermentations. Studies with different concentrations of LB media were also conducted to determine the optimal concentration for use during large scale production. Briefly, the six strains were grown in 500 mL and 1 L flasks in LB medium at concentrations of 100, 50, 25, 15, and 10% LB for the purpose of finding the effect of nutrients in the fermenter to induce spore formation. The 10 L fermenter trials were done with 10% LB which induced spore formation within 18 h of fermentation when the nutrients were limiting.

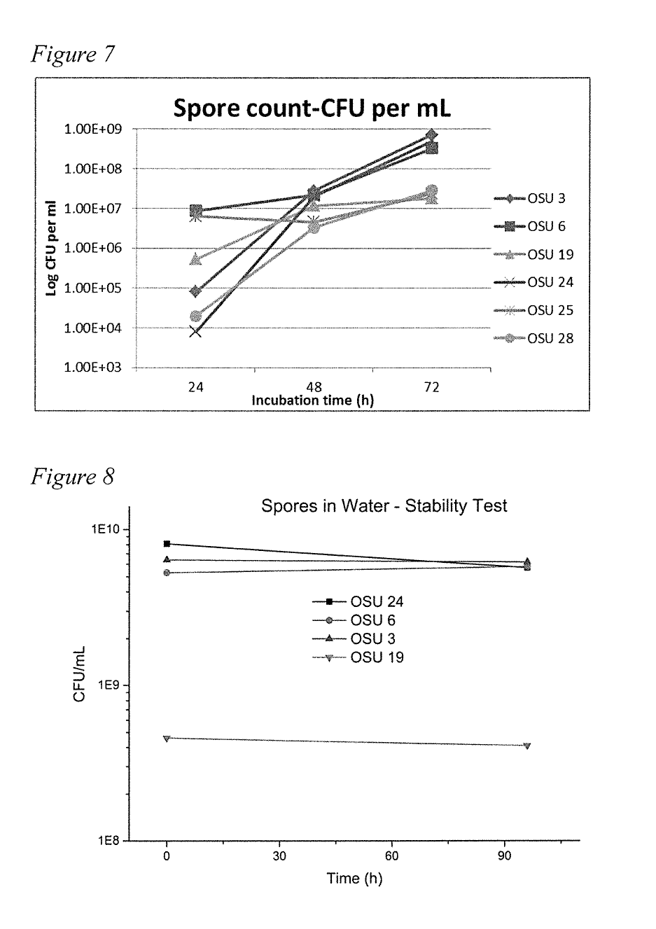

The kinetics of each strain was calculated based on total plate count (vegetative cells) and spores recovered reported as colony forming units per mL (CFU/mL) as a function of time (FIG. 7). As can be seen, at 24 h, the production of spores differs about three log cycles among the strains. At 72 h, strains OSU 3, 24, and 6 separate in a group with about one log higher spore counts than OSU 19, 28, and 25. Overall, 10⁷ and 10⁸ are good spore production values and all the strains tested were deemed acceptable to continue in the selected group.

The spores obtained from the selected group were also analyzed for their survival in water with the objective of testing if it would be possible to add the spores to water as well as feed. Examples of spore survival in water after 92 h at 25° C. incubation are shown in FIG. 8. As can be seen, the four strains showed only a slight decrease in CFU/mL after 92 h in water, suggesting they could be used in the water for at least that period of time.

Finally, compatibility studies were performed to learn which, if any, of the strains interfered with the growth of one or more other strains on LB agar (not shown).

### Example 3

### Selection of Three Strains for In Vivo Efficiency Test

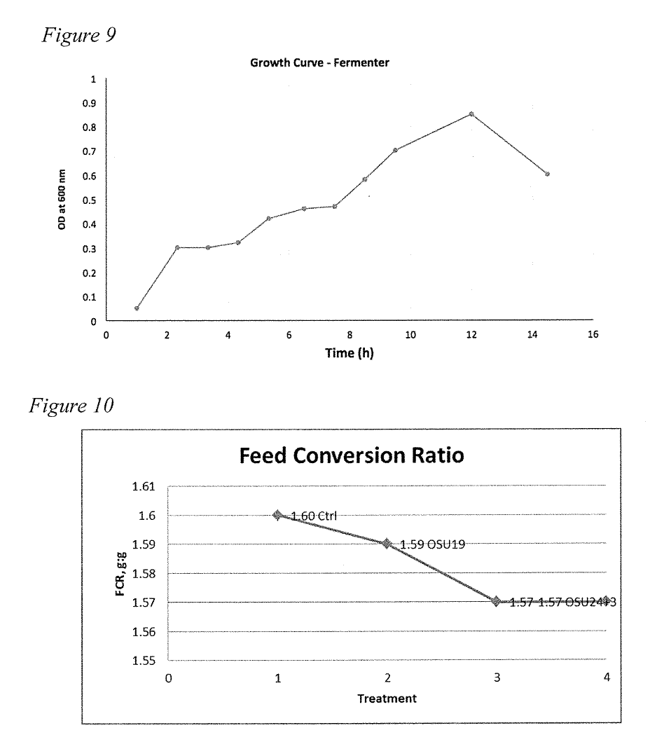

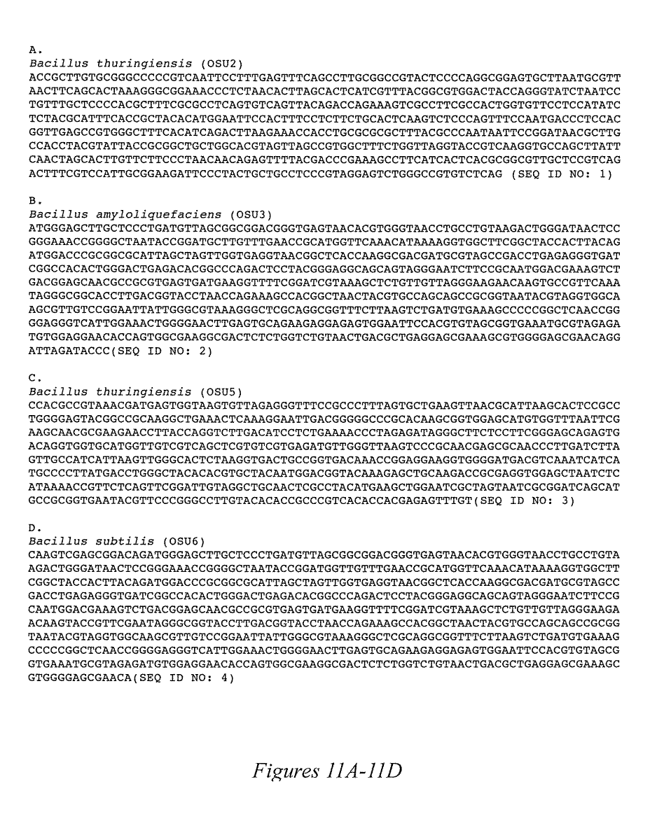

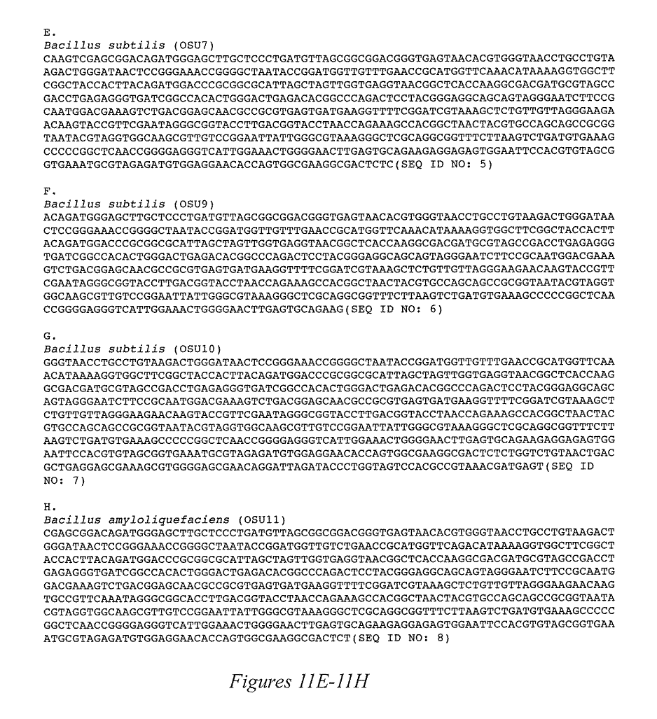

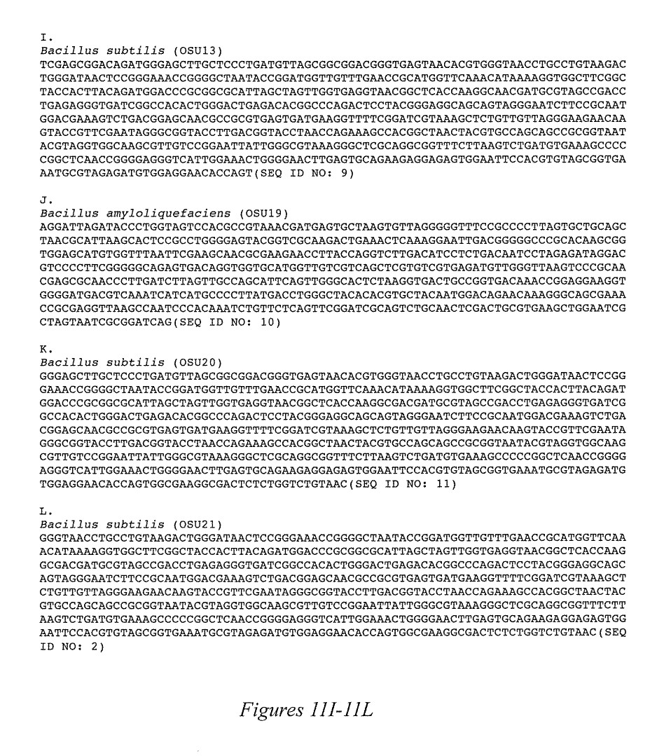

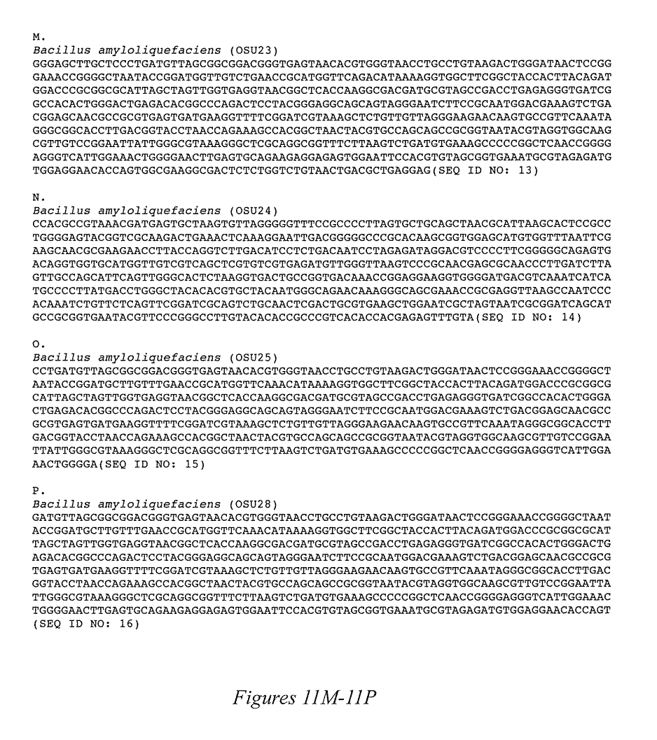

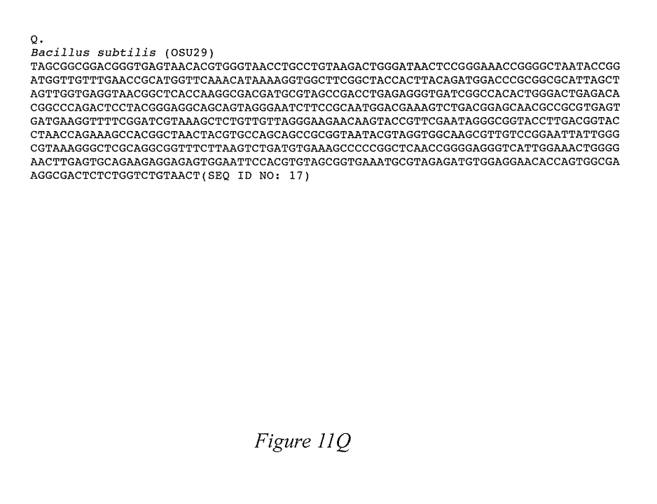

Based on the foregoing tests, three strains, denominated OSU 3, 19, and 24, (NRLL numbers B-67040, B-67048 and B-67052, respectively), were selected for in vivo testing. The selection of strains was based on growth, production of spores, and compatibility. As expected, there was a difference in the growth of the selected strains in a 10 L fermenter compared to growth in 1 L flasks. The difference is attributed to the limitation of air during the incubation in flasks. In the fermenters, air and agitation are delivered at a higher rate, thus affecting the growth. Consequently, cultures reached their maximum growth (as estimated by optical density at 600 nm (OD 600)) more quickly. The strains were grown at 10% LB and the spores were recovered by centrifugation at 5,000×g for 5 min. A representative sample of the growth curve of one strain, “OSU 3” is presented in FIG. 9.

A summary of the production of Bacillus spores is presented in Table 2. From three strains, OSU 3, 19, and 24, the recovered yield of spores ranged from 16,800 to 330,000 doses available in the total volume that they were concentrated in. From the total number of spores, the calculated dose for a concentration of 1.2×10⁶ is represented in the last column of total doses available for the in vivo trial.

TABLE 2
Summary of total spores recovered from a 10 L fermenter and
calculated for a dose of 1.2 million (10⁶) spores.

| OSU | Spores in | | |
|---|---|---|---|
| Strain | concentrate | Spores/dose | Doses/total concentrate |
| 3 | 1.87E+11 | 1.20E+06 | 156,000 |
| 6 | 8.52E+07 | 1.20E+06 | 71 |
| 19 | 2.03E+10 | 1.20E+06 | 16,875 |
| 24 | 3.96E+11 | 1.20E+06 | 330,000 |

In previous compatibility studies, strain 19 had shown limited compatibility with strains 3 and 24, so in vivo, strain 19 was tested by itself and strains 24+3 were tested as a cocktail.

The poultry study included testing three different treatment groups and one untreated control group. The treatments were 1) a cocktail treatment in which 10⁶ spores of the two Bacillus strains, OSU 24 and OSU3, were administered orally twice per week; 2) a second treatment in which 10⁶ spores of a single Bacillus strain (OSU 19) were administered orally twice per week and 3) a dry feed preparation of higher dose which delivered 10⁶ spores per gram of dry feed of a preparation of OSU strain 3 and 24. For the latter treatment, the spores were suspended in sterile water, sprayed on the dry feed, and blended in an auger bucket-type mixer. The total number of chickens was 180 birds with ten replicates (cages) per treatment with each cage containing 6 chickens for the oral doses (10⁶ spores twice per week) and the control group, and 60 birds for the high dose (10⁶ spores per gram of dry feed). In summary, there was one control untreated group, two oral dose treatments and one dry feed treatment.

Dilutions of the 3 and 24 strains were prepared such that a suspension of 250 uL delivered 1.2×10⁶ spores of each strain when delivered orally with a total final volume of 500 uL. Dilutions for strain 19 were also made to deliver 1.2×10⁶ spores per 500 uL. The oral dose represented actual spores delivered to each chicken; thus, the number of spores actually consumed by each chicken was known. It is well known that, in animal studies using dry food, accounting for feed waste is a challenge when determining the actual amount of feed that is ingested. The dry feed treatment was prepared so as to deliver a concentration of 5×10⁶ spores per gram of dry feed. This represents a convenient industry application and allowed direct comparison with the oral doses.

60 chickens (male Cobb 500 broilers) per treatment and control groups were tested during the 42 days of the vivo study. The results are presented in FIG. 10. While there were no statistical differences in FCR due to the treatment with Bacillus spp. probiotic preparations, numerically the mean values of FCR trended lower, showing a decrease from 1.60 for the control group to 1.59 and 1.57 for the OSU 19 and OSU 24+3 treatment groups, respectively. These results suggest an improvement in the digestion efficiency. Such improvements translate directly into savings in feed consumption, one of the most important inputs in live weight animal production. The results showed significantly lower feed consumption at the starter phase by the broilers at day 14 for the probiotic treatments compared to the control. The feed consumption for the control was 446 g while for OSU 19 was 399 g, for OSU 3+24 oral dose 416 g and for the OSU 3+24 in the dry feed 409 g. In the starter phase the feed diet has higher protein content compared to the next two periods of grower and finisher feed diets. Thus specific combination of strains are investigated tailored to the diet composition at specific growth phases of the broilers.

### Example 4

### Further Investigations

The effect of the dose level of Bacillus spp. spores is evaluated with a higher number of experimental units (cages of 6 chickens per cage). Growth and spore production of cocktails prepared with three or more strains, including other strains selected via in vitro studies, are evaluated.

The effects of increased dose levels of Bacillus spores (e.g. from 10⁶ to 10¹⁰ and 10¹² per gram of feed) incorporated into dry feed is analyzed and FCR is determined.

The number of experimental units is increased by a factor of 2.4. Experimental units are cages (cages with 6 chickens each) per treatment; the increase is from 10 cages (10 cages with 6 chickens per cage=60 chickens total) utilized up to this point to 24 cages (24 cages with 6 chickens per cage=144 chickens).

Additional strains are selected for testing. Three or more strains from Bacillus amyloquefaciens, B. subtilis and B. thuringiensis are included, based on their enzymatic activity and growth kinetics. Synergistic effects are observed with cocktails comprising these and previously tested strains.

The effect of higher temperature on the survival of spores is examined. For example, the effects of summer temperatures on a commercial farm (e.g. from about 32 to about 45° C.) are investigated.

The effect of probiotic preparations and dose on specific growth periods is examined. For example, the effects of such preparations on grower, intermediate, and finisher phases where the protein content of the feed are specific for each growing phases are investigated.

The effect of probiotic preparations and dose on the performance and FCR of stressed animals is examined. For example, the effect of such preparations on broilers subjected to ambient growing temperatures of 33° C. from day 28 to 42 days is investigated.

### REFERENCES

ALMEIDA, F. N., SULABO, R. C. & STEIN, H. H. 2013. Effects of a novel bacterial phytase expressed in Aspergillus Oryzae on digestibility of calcium and phosphorus in diets fed to weanling or growing pigs. J Anim Sci Biotechnol, 4, 8.
ALEXOPOULOS, C., GEORGOULAKIS, I. E., TZIVARA, A., KYRIAKIS, C. S., GOVARIS, A., & KYRIAKIS, S. C. 2004. Field evaluation of the effect of a probiotic containing Bacillus licheniformis and Bacillus subtilis spores on the health status, performance, and carcass quality of grower and finisher pigs. J Vet Med A Physiol Pathol Clin Med 51:306-312. doi: 10.1111/j.1439-0442.2004.00637
CASULA, G., & CUTTING, S. M. 2002. Bacillus probiotics: Spore germination in the gastrointestinal tract. Appl Environ Microbiol. 68(5): 2344-2352.
CUTTING, S. M. 20 II. Bacillus probiotics. Food Microbiology, 28, 214-220.
FAN, Y. X., TIAN, Y. 1., ZHAO, X. Y., ZHANG, J. X. & LIU, J. L. 2013. Isolation of acetoin-producing Bacillus strains from Japanese traditional food natto. Preparative Biochemistry & Biotechnology, 43, 551-564.
FARHAT-KHEMAKHEM, A., BEN ALI, M., BOUKHRIS, I., KHEMAKHEM, B., MAGUIN, E., BEJAR, S. & CHOUA YEKH, H. 2013. Crucial role of Pro 257 in the thermostability of Bacillus phytases: Biochemical and structural investigation. International Journal of Biological Macromolecules, 54, 9-15.
FU, S., SUN, J., QIAN, L. & LI, Z. 2008. Bacillus phytases: Present scenario and future perspectives. Applied Biochemistry and Biotechnology, 151, 1-8.
FULLER, R. 1989. Probiotics in man and animals. J. Appl. Bacteriol. 66: 365-378. GAGGIA, F., MATTARELLI, P. & BIAVATI, B. 2010. Probiotics and prebiotics in animal feeding for safe food production. International Journal of Food Microbiology, 141, Supplement, S15-S28.
GILLILAND, S. E., STALEY, T. E., & BUSH, L. J. 1984. Importance of bile tolerance of lactobacillus-acidophilus used as a dietary adjunct. Journal of Dairy Science 67, 3045.
GUO X, LI D, LU W, PIAO X, & CHEN X. 2006. Screening of Bacillus strains as potential probiotics and subsequent confirmation of the in vivo effectiveness of Bacillus subtilis MA139 in pigs. Antonie Van Leeuwenhoek. 90(2):139-46. Epub 2006 Jul. 4.
HE, S. X., ZHANG, Y., XU, L., YANG, Y. L., MARUBASHI, T., ZHOU, Z. G. & YAO, B. 2013. Effects of dietary Bacillus subtilis C-31 02 on the production, intestinal cytokine expression and autochthonous bacteria of hybridtilapia Oreochromis niloticus female x Oreochromis aureus male. Aquaculture, 412, 125-130.
HONG H. A., DUC L. H., & CUTTING S. M. 2005. The use of bacterial spore formers as probiotics. FEMS Microbiol Rev 29:813-835. doi:10.1016/j.
KNOWLTON, K. F., RADCLIFFE, J. S., NOVAK, C. L. & EMMERSON, D. A. 2004. Animal management to reduce phosphorus losses to the environment. J Anim Sci, 82, E173-E195.
LEE, J., PARK, 1., CHOI, Y. & CHO, J. 2012. Bacillus strains as feed additives: In vitro evaluation of its potential probiotic properties. Revista Colombiana De Ciencias Pecuarias, 25, 577-585.
LIPPOLIS, R., SICILIANO, R. A., MAZZEO, M. F., ABBRESCIA, A., GNONI, A., SARDANELLI, A. M. & PAPA, S. 2013. Comparative secretome analysis off our isogenic Bacillus clausii probiotic strains. Proteome Science, 11, 28. Website at www.proteomesci.com/content/11/1/28
MURUGESAN, G. R., ROMERO, L. F., & PERSIA, M. E. 2014. Effects of Protease, Phytase and a Bacillus sp. Direct-Fed Microbial on Nutrient and Energy Digestibility, Ileal Brush Border Digestive Enzyme Activity and Cecal Short-Chain Fatty Acid Concentration in Broiler Chickens. PLoS ONE 9(7): e101888. doi:10.1371/joumal.pone.010188. Published July 2014.
QIN, H. B., YANG, H. 1., QIAO, Z. 1., GAO, S. S. & LIU, Z. 2013. Identification and characterization of a Bacillus subtilis strain HB-I isolated from Yandou, a fermented soybean food in China. Food Control, 31, 22-27.
QUIGLEY, E. M. M. 20 IO. Prebiotics and probiotics; modifying and mining the microbiota. Pharmacological Research, 61, 213-218.
SANTOSO, U., TANAKA, K., OHTANI, S., & SAKAIDA, M. 2001. Effect of Fermented Product from Bacillus Subtilis on Feed Conversion Efficiency, Lipid Accumulation and Ammonia Production in Broiler Chicks. Asian-Austr J Anim Sci 14: 333-337.
SEN. S., INGALE, S. L., KIM, Y. W., KIM, J. S., KIM, K. H., LOHAKARE, J. D., KIM, E. K., KIM, H. S., RYU, M. H., KWON, I. K., & CHAE, B. J. 2012. Effect of supplementation of Bacillus subtilis LS 1-2 to broiler diets on growth performance, nutrient retention, caecal microbiology and small intestinal morphology. Research in Veterinary Science 93, 264-268.
SHIVARAMAIAH, S., PUMFORD, N. R., MORGAN, M. J., WOLFENDEN, R. E., WOLFENDEN, A. D., TORRES-RODRIGUEZ, A., HARGIS, B. M., & TELLEZ, G. 2011. Evaluation of Bacillus species as potential candidates for direct-fed microbials in commercial poultry. Poultry Sci. 90(7): 1574-1580 DOI: 10.3382/ps.2010-00745 Published: Jul. 1 2011.
TAM, N. K. M., UYEN, N. Q., HONG, H. A., DUC, L. H., HOA, T. T., SERRA, C. R., HENRIQUES, A. O. & CUTTING, S. M. 2006. The intestinal life cycle of Bacillus subtilis and close relatives. Journal of Bacteriology, 188, 2692-2700.
ZHU, H., TIAN, B. Z., LIU, W., ZHANG, S. S., CAO, C. X., ZHANG, Y. & ZOU, W. S. 2012. A three-stage culture process for improved exopolysaccharide production by Tremella fuciformis. Bioresource Technology, 116, 526-528.
ZHU, Y. P., FAN, J. F., CHENG, Y. Q. & LI, L. T. 2008. Improvement of the antioxidant activity of Chinese traditional fermented okara (Meitauza) using Bacillus subtilis B2. Food Control, 19, 654-661.

1. A probiotic composition comprising endospores of one or more Bacillus species deposited under NRRL accession number B-67039, B-67040, B-67041, B-67042, B-67043, B-67044, B-67045, B-67046, B-67047, B-67048, B-67049, B-67050, B-67051, B-67052, B-67053, B-67054, or B-67055, and a physiologically acceptable carrier.

2. The probiotic composition of claim 1, wherein enzyme activities of said one or more Bacillus species include at least one of phytase activity, α-amylase activity, cellulolytic activity and protease activity.

3. The probiotic composition of claim 1, wherein said one or more Bacillus species are bile acid resistant.

4. The probiotic of claim 1, wherein said endospores of said one or more Bacillus species remain viable at 80° C.

5. A method of increasing nutrient availability from a high fiber diet and digestion efficiency in a host in need thereof, comprising administering to said host a probiotic composition comprising endospores of one or more Bacillus species deposited under NRRL accession number B-67039, B-67040, B-67041, B-67042, B-67043, B-67044, B-67045, B-67046, B-67047, B-67048, B-67049, B-67050, B-67051, B-67052, B-67053, B-67054, or B-67055, and a physiologically acceptable carrier.

6. The method of claim 5, wherein enzyme activities of said one or more Bacillus species or strains include at least one of phytase activity, α-amylase activity, cellulolytic activity and protease activity.

7. The method of claim 5, wherein said one or more Bacillus species or strains are bile acid resistant.

8. The method of claim 5, wherein said endospores of said one or more Bacillus species or strains remain viable at 80° C.

9. A method of increasing weight gain in an animal in need thereof, comprising administering to said animal a probiotic composition comprising endospores of one or more Bacillus species deposited under NRRL accession number B-67039, B-67040, B-67041, B-67042, B-67043, B-67044, B-67045, B-67046, B-67047, B-67048, B-67049, B-67050, B-67051, B-67052, B-67053, B-67054, or B-67055, and a physiologically acceptable carrier.

10. The method of claim 9, wherein enzyme activities of said one or more Bacillus species or strains include at least one of phytase activity, α-amylase activity, cellulolytic activity and protease activity.

11. The method of claim 9, wherein said one or more Bacillus species or strains are bile acid resistant.

12. The method of claim 9, wherein said endospores of said one or more Bacillus species or strains remain viable at 80° C.

13. The method of claim 9, wherein said animal is a chicken.

14. A probiotic food product comprising endospores of one or more Bacillus species deposited under NRRL accession number B-67039, B-67040, B-67041, B-67042, B-67043, B-67044, B-67045, B-67046, B-67047, B-67048, B-67049, B-67050, B-67051, B-67052, B-67053, B-67054, or B-67055.

15. The probiotic food product of claim 14, wherein said probiotic food product is feed for an animal.

16. The probiotic food product of claim 14, wherein said animal is a chicken.

17. A probiotic formulation, comprising one or more Bacillus spores isolated from sourdough, and ingestible food product which is not sourdough.

18. A method of making a probiotic food product comprising combining endospores of one or more Bacillus species deposited under NRRL, accession number B-67039, B-67040, B-67041, B-67042, B-67043, B-67044, B-67045, B-67046, B-67047, B-67048, B-67049, B-67050, B-67051, B-67052, B-67053, B-67054, or B-67055, with one or more additional nutritional components or carriers.

19. The probiotic of claim 2, wherein said endospores of said one or more Bacillus species remain viable at 80° C.

20. The probiotic of claim 3, wherein said endospores of said one or more Bacillus species remain viable at 80° C.
